(19) United States
(12) Patent Application Publication
(10) Pub. No.: US 2021/0404459 A1
(43) Pub. Date: Dec. 30, 2021

(54) PERISTALTIC PUMP APPARATUS FOR USE WITH BIOPSY SAMPLING DEVICES

(71) Applicant: Bard Peripheral Vascular, Inc., Franklin Lakes, NJ (US)

(72) Inventors: Brian Boyle, Chandler, AZ (US); Bryon Pelzek, Gold Canyon, AZ (US); Ranjani Sampath Kumaran, Tempe, AZ (US)

(21) Appl. No.: 16/911,590

(22) Filed: Jun. 25, 2020

(51) Int. Cl. F04B 43/00; F04B 43/12; F04B 49/06; A61B 10/02; A61M 19/00

(52) U.S. Cl. CPC F04B 43/0081; F04B 43/1253; F04B 49/06; A61B 10/0283; A61M 2205/103; A61M 2205/50; A61M 2205/52; A61B 2010/0208; A61M 19/00

(57) ABSTRACT

A peristaltic pump apparatus for a medical device has a housing, a motor, a first peristaltic pump head, a second peristaltic pump head, a first clutch assembly, and a second clutch assembly. The motor is directly connected to the housing and has a rotatable drive shaft. Each of the first peristaltic pump head and the second peristaltic pump head is coupled to the rotatable drive shaft. The first clutch assembly has a first rotor fixedly connected to the rotatable drive shaft and a first stator directly connected to the housing. The first clutch assembly is configured to selectively electromagnetically couple the first rotor to the first peristaltic pump head. The second clutch assembly has a second rotor fixedly connected to the rotatable drive shaft and a second stator directly connected to the housing. The second clutch assembly selectively electromagnetically couples the second rotor to the second peristaltic pump head.

### CROSS-REFERENCE TO RELATED APPLICATIONS

None.

### TECHNICAL FIELD

The present invention relates generally to pumps for use with medical instruments, and, more particularly, to peristaltic pumps for use with medical instruments to acquire and transport tissue from a target site.

### BACKGROUND ART

During breast biopsies it is essential to be able to transport a biopsy sample after cutting the sample from the patient. Currently, vacuum pumps with a vacuum reservoir are used to transport cut samples from a distal end of a biopsy device to a sample collection basket. However, during use, vacuum pumps with vacuum reservoirs create uncomfortable loud vibrations in an exam room. Furthermore, vacuum reservoirs are required for each such system and need to be recharged after each loss of vacuum. Thus, vacuum pumps cut on and off during medical procedures to recharge the vacuum reservoir after loss of vacuum.

Moreover, because vacuum discharge occurs numerous times during a procedure, vacuum pumps with vacuum reservoirs cause delays during medical procedures as the user is required to recharge the vacuum reservoir before continuing to collect samples from the patient.

In addition, complicated pinch valve assemblies are required within a vacuum pump and vacuum reservoir system to charge and discharge the vacuum reservoir. Furthermore, vacuum pumps can only hold a static vacuum.

### SUMMARY OF INVENTION

The present invention provides a peristaltic pump apparatus for use with a medical device.

The invention in one form is directed to a peristaltic pump apparatus that has a housing and a motor directly connected to the housing. The motor has a rotatable drive shaft. A first peristaltic pump head is coupled to the rotatable drive shaft. A second peristaltic pump head is coupled to the rotatable drive shaft. The peristaltic pump apparatus also has a first clutch assembly and a second clutch assembly. The first clutch assembly has a first rotor that is fixedly connected to the rotatable drive shaft and a first stator directly connected to the housing. The first clutch assembly is configured to selectively electromagnetically couple the first rotor to the first peristaltic pump head. The second clutch assembly has a second rotor that is fixedly connected to the rotatable drive shaft and a second stator fixedly connected to this housing. The second clutch assembly is configured to selectively electromagnetically couple the second rotor to the second peristaltic pump head.

The invention in another form is directed to a biopsy system. The biopsy system includes a biopsy sampling device and a peristaltic pump apparatus. The peristaltic pump apparatus is coupled in fluid communication with the biopsy sampling device. The peristaltic pump apparatus includes a housing, a controller circuit, and a motor that is electrically coupled to the controller circuit. The motor, which is directly connected to the housing, has a rotatable drive shaft. The peristaltic pump apparatus has a first peristaltic pump head and a second peristaltic pump head. The first peristaltic pump head is coupled to the rotatable drive shaft, and the second peristaltic pump head is coupled to the rotatable drive shaft.

An advantage of the present invention is the peristaltic pump apparatus includes a first clutch assembly and a second clutch assembly. The first clutch assembly has a first rotor that is fixedly connected to the rotatable drive shaft and a first stator that is directly connected to the housing. The first clutch assembly is electrically coupled to the controller circuit. The first clutch assembly is configured to selectively electromagnetically couple the first rotor to the first peristaltic pump head. The second clutch assembly has a second rotor that is fixedly connected to the rotatable drive shaft and a second stator that is directly connected to the housing. The second clutch assembly is electrically coupled to the controller circuit. The second clutch assembly is configured to selectively electromagnetically couple the second rotor to the second peristaltic pump head.

Yet another advantage is the controller circuit is configured to selectively supply electrical control signals to each of the motor, the first clutch assembly, and the second clutch assembly.

The invention in one form is directed to a biopsy system. The biopsy system includes a biopsy sampling device and a peristaltic pump apparatus. The biopsy sampling device includes a cannula, a third port, a fourth port, and a sampling basket. The cannula has a distal sampling end. The sampling basket is disposed between the distal sampling end of the cannula and the third port. The sampling basket is distal of the third port. The fourth port is disposed between the distal sampling end of the cannula and the sampling basket. The fourth port is distal of the third port.

An advantage of the present invention is the peristaltic pump apparatus is coupled in fluid communication with the biopsy sampling device. The peristaltic pump apparatus includes a housing, a controller circuit, a motor having a rotatable drive shaft, a first fluid reservoir having a first port, and a second fluid reservoir having a second port. The motor is directly connected to the housing. The peristaltic pump apparatus further includes a first conduit and a second conduit. The first conduit has a first conduit distal end and a first conduit proximal end. The first conduit proximal end is directly connected to the first port. The first conduit distal end is directly connected to the third port. The second conduit has a second conduit distal end and a second conduit proximal end. The second conduit proximal end is directly connected to the second port. The second conduit distal end is directly connected to the fourth port.

The peristaltic pump apparatus includes a first peristaltic pump head, which is coupled to the rotatable drive shaft, and a second peristaltic pump head, which is coupled to the rotatable drive shaft. The first peristaltic head has a first outermost perimeter, and the second peristaltic pump head has a second outermost perimeter. At least three first idle rollers are connected to the first peristaltic pump head and are angularly spaced around the first outermost perimeter. At least three second idle rollers are connected to the second peristaltic pump head and are angularly spaced around the second outermost perimeter.

Another advantage is that the peristaltic pump apparatus includes a first clutch assembly and a second clutch assembly. The first clutch assembly has a first rotor that is fixedly connected to the rotatable drive shaft and a first stator that is directly connected to the housing. The first clutch assembly is electrically coupled to the controller circuit. The first clutch assembly is configured to selectively electromagnetically couple the first rotor to the first peristaltic pump head. The second clutch assembly has a second rotor that is fixedly connected to the rotatable drive shaft and a second stator that is directly connected to the housing. The second clutch assembly is electrically coupled to the controller circuit. The second clutch assembly is configured to selectively electromagnetically couple the second rotor to the second peristaltic pump head.

Yet another advantage is the controller circuit has a processor circuit and a memory circuit. The processor circuit is configured to execute motor program instructions to control the rotation of the rotatable drive shaft of the motor. The processor circuit is configured to execute first clutch assembly program instructions to selectively electromagnetically couple the first rotor to the first peristaltic pump head during a first period of engagement. The processor circuit is configured to execute second clutch assembly program instructions to selectively electromagnetically couple the second rotor to the second peristaltic pump head during a second period of engagement.

Advantageously, at least two of the at least three first idle rollers contact the first conduit during the first period of engagement, and at least two of the at least three second idle rollers contact the second conduit during the second period of engagement. Furthermore, the first peristaltic pump head, the first conduit, and the first fluid reservoir are arranged to prevent a positive pressure fluid flow in a distal direction and to create a vacuum that flows in a proximal direction toward the first fluid reservoir.

### BRIEF DESCRIPTION OF DRAWINGS

The above-mentioned and other features and advantages of this invention, and the manner of attaining them, will become more apparent and the invention will be better understood by reference to the following description of embodiments of the invention taken in conjunction with the accompanying drawings, wherein:

FIG. 1 is a schematic representation of the biopsy system having a peristaltic pump apparatus and a biopsy sampling device;

FIG. 2 is a schematic representation of the peristaltic pump apparatus;

FIG. 3 is a schematic representation of the motor, the rotatable drive shaft, the plurality of clutch assemblies, the plurality of peristaltic pump heads, and the plurality of conduits;

FIG. 4 is a cross-section of the motor, the rotatable drive shaft, the plurality of clutch assemblies, and the plurality of peristaltic pump heads of FIG. 3;

FIG. 5 is a graph depicting the delivery of various fluids by an exemplary embodiment of the peristaltic pump apparatus over time;

FIG. 6A shows a cross-section taken along line 6A of FIG. 3 showing the first peristaltic pump head and the first conduit;

FIG. 6B shows a cross-section taken along line 6B of FIG. 3 showing the second peristaltic pump head and the second conduit;

FIG. 6C shows a cross-section taken along line 6C of FIG. 3 showing the third peristaltic pump head and the third conduit;

Corresponding reference characters indicate corresponding parts throughout the several views. The exemplifications set out herein illustrate at least one embodiment of the invention, and such exemplifications are not to be construed as limiting the scope of the invention in any manner.

### DESCRIPTION OF EMBODIMENTS

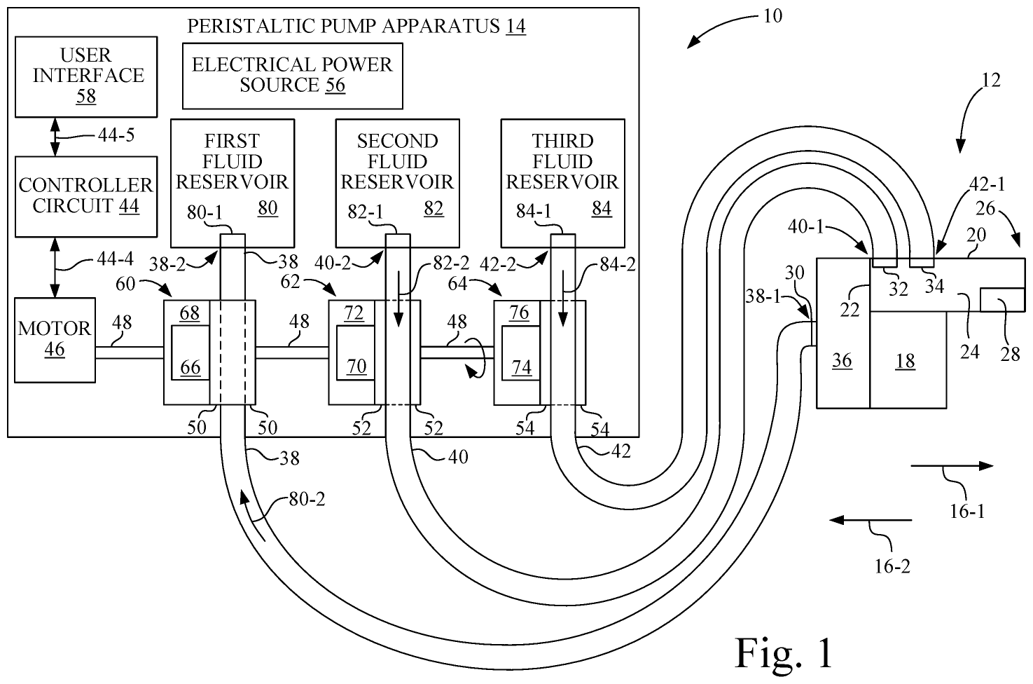

Referring now to the drawings, and more particularly to FIG. 1, there is shown a biopsy system 10 which includes a biopsy sampling device 12 and a peristaltic pump apparatus 14. FIG. 1 shows the distal direction 16-1 and the proximal direction 16-2.

The biopsy sampling device 12 generally includes a non-invasive, e.g., non-disposable driver assembly 18 and an invasive, e.g., disposable, cannula 20. As used herein, the term “non-disposable” is used to refer to a device that is intended for use on multiple patients during the lifetime of the device, and the term “disposable” is used to refer to a device that is intended to be disposed of after use on a single patient. Cannula 20 has a cannula proximal end 22, a cannula lumen 24, and a distal sampling end 26 with a sampling notch 28, which is used for severing tissue from a target site in a patient. Biopsy sampling device 12 also has a plurality of ports, such as a third port 30 and a fourth port 32, and a sixth port 34. Biopsy sampling device 12 includes a sampling basket 36 that is disposed between distal sampling end 26 of cannula 20 and third port 30. Sampling basket 36 is distal of third port 30, and fourth port 32 is disposed between distal sampling end 26 of cannula 20 and sampling basket 36. As shown in FIG. 1, fourth port 32 is distal of third port 30. Sixth port 34 is on cannula 20 and is positioned between sampling basket 36 and distal sampling end 26.

Peristaltic pump apparatus 14 is coupled in fluid communication with biopsy sampling device 12 via a plurality of flexible conduits, e.g., a first conduit 38, a second conduit 40, a third conduit 42, etc. FIG. 1 shows peristaltic pump apparatus 14 includes first conduit 38 having a first conduit distal end 38-1 and a first conduit proximal end 38-2, second conduit 40 having a second conduit distal end 40-1 and a second conduit proximal end 40-2, and third conduit 42 having a third conduit distal end 42-1 and a third conduit proximal end 42-2.

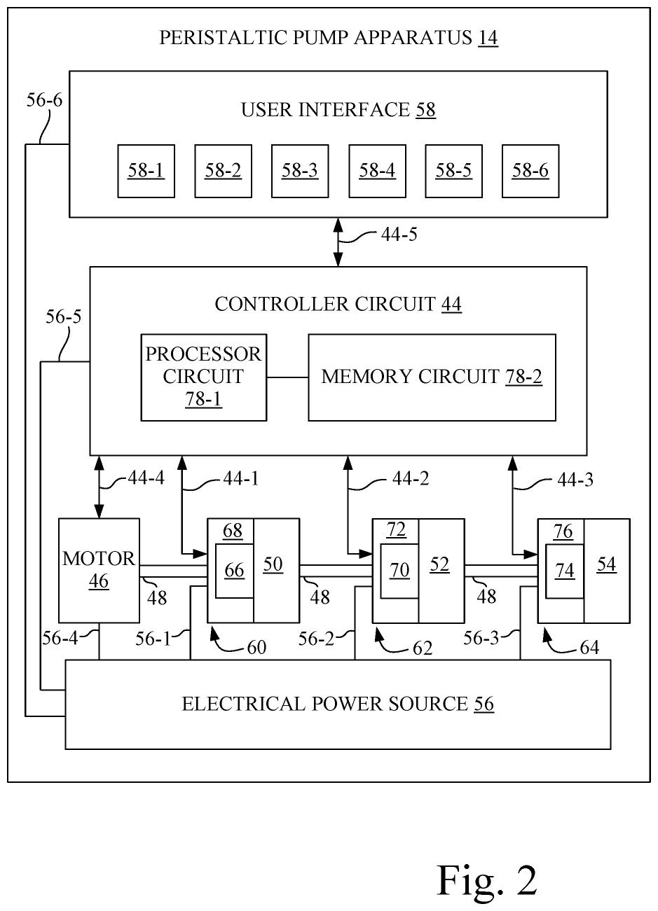

Peristaltic pump apparatus 14 includes a housing 43, a controller circuit 44, a motor 46 having a rotatable drive shaft 48, and a plurality of pump heads, e.g., a first peristaltic pump head 50, a second peristaltic pump head 52, a third peristaltic pump head 54. As best seen in FIG. 2, peristaltic pump apparatus 14 is connected to an electrical power source 56, such as, for example, a battery or connections to the power grid. As shown in FIG. 1 and FIG. 2, peristaltic pump apparatus 14 includes a user interface 58. Peristaltic pump apparatus 14 includes a plurality of clutch assemblies, e.g., a first clutch assembly 60, a second clutch assembly 62, and a third clutch assembly 64.

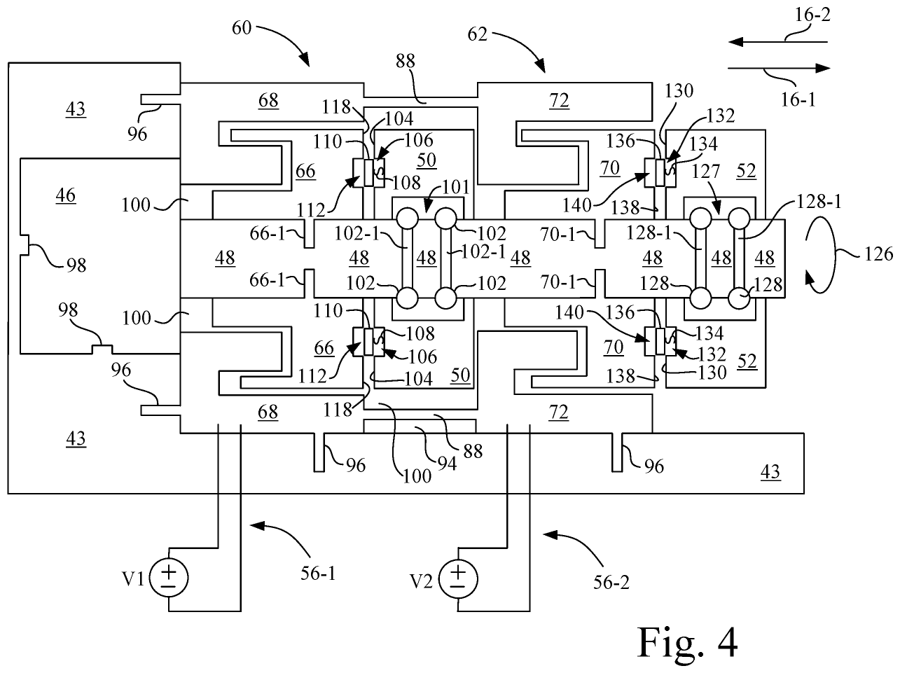

Each clutch assembly includes a rotor and a corresponding stator. Each stator is an electromagnetic coil that remains stationary and on the outside of the corresponding rotor that is fixedly connected to rotatable drive shaft 48 of motor 46 via a mechanical coupler, such as, e.g., a set screw, a weld, etc. In particular, first clutch assembly 60 includes a first rotor 66 and a first stator 68. First rotor 66 is fixedly connected to the rotatable drive shaft 48 of motor 46 via a first mechanical coupler 66-1 as shown in FIG. 4. Second clutch assembly 62 includes a second rotor 70 and a second stator 72. Second rotor 70 is fixedly connected to the rotatable drive shaft 48 of motor 46 via a second mechanical coupler 70-1 as shown in FIG. 4. Third clutch assembly 64 includes a third rotor 74 and a third stator 76, and third rotor 74 is fixedly connected to the rotatable drive shaft 48 via third mechanical coupler 74-1 as shown in FIG. 4. Each rotor rotates as motor 46 rotates the rotatable drive shaft 48.

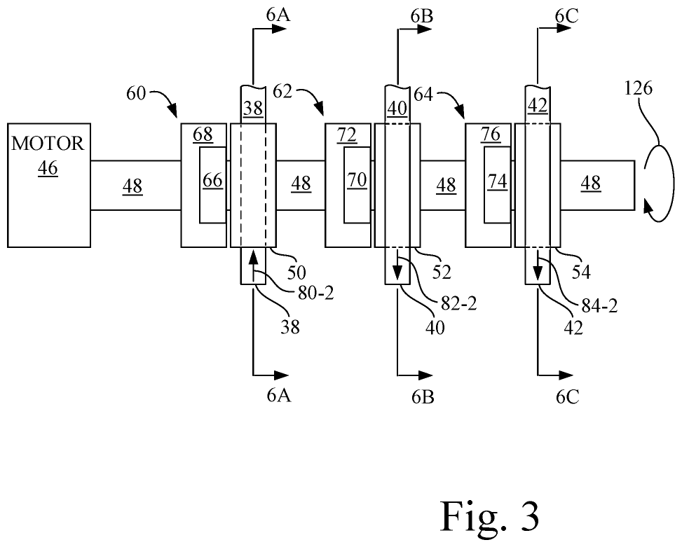

Electrical power source 56 provides electrical power to all electrically powered components of peristaltic pump apparatus 14 by one or more electrical connections made up of electrical conductors, e.g., wires or circuit traces. FIG. 1 and FIG. 3 do not include electrical connections from electrical power source 56 for simplicity and readability. As shown in FIG. 2, electrical power source 56 is electrically connected to first clutch assembly 60, in particular, to first stator 68, via first electrical connection 56-1. Electrical power source 56 is electrically connected to second clutch assembly 62, in particular, to second stator 72, via second electrical connection 56-2. Electrical power source 56 is electrically connected to third clutch assembly 64, in particular, to third stator 76, via third electrical connection 56-3. Electrical power source 56 is electrically connected to motor 46 via fourth electrical connection 56-4. Electrical power source 56 is electrically connected to controller circuit 44 via fifth electrical connection 56-5. Electrical power source 56 is electrically connected to user interface 58 via sixth electrical connection 56-6.

Controller circuit 44 may be assembled on an electrical circuit board and includes, for example, a processor circuit 78-1 and a memory circuit 78-2. Processor circuit 78-1 has one or more programmable microprocessors and associated circuitry, such as an input/output interface, clock, buffers, memory, etc. Memory circuit 78-2 is communicatively coupled to processor circuit 78-1, e.g., via a bus circuit, and is a non-transitory electronic memory that may include volatile memory circuits, such as random access memory (RAM), and non-volatile memory circuits, such as read only memory (ROM), electronically erasable programmable ROM (EEPROM), NOR flash memory, NAND flash memory, etc. Controller circuit 44 may be formed as one or more Application Specific Integrated Circuits (ASIC).

Controller circuit 44 is electrically and communicatively coupled to each of the following: first stator 68 via first communication link 44-1, second stator 72 via second communication link 44-2, third stator 76 via third communication link 44-3, motor 46 via fourth communication link 44-4, and user interface 58 via fifth communication link 44-5. Each of the control circuit's communication links 44-1, 44-2, 44-3, 44-4, and 44-5 is bi-directional and may be wired or wireless. Controller circuit 44 is configured to selectively supply electrical control signals to each of motor 46, first clutch assembly 60, second clutch assembly 62, and third clutch assembly 64. Although FIG. 1 and FIG. 3 do not show first communication link 44-1, second communication link 44-2, or third communication link 44-3 for the sake of simplicity and readability, first communication link 44-1, second communication link 44-2, and third communication link 44-3 are included in FIG. 2.

Still referring to FIG. 2, user interface 58 includes control buttons, such as vacuum control button 58-1, and visual indicators. Control buttons may include, for example, vacuum control button 58-1, anesthetic control button 58-2, and saline control button 58-3. Control buttons, such as, e.g., vacuum control button 58-1, may be physical buttons or icons on a touchscreen. Control buttons provide control over various functions of the peristaltic pump apparatus 14. User interface 58 may also include one or more motor control buttons 58-4 to control whether the motor is on or off. Control buttons may include a visual indicator on a display screen 58-5 and/or one or more light emitting diodes (LED) 58-6. Control buttons may include tactile feedback to the user when activated.

Motor 46 may be an electrical motor, such as, for example, a direct current (DC) motor, stepper motor, etc. Motor 46 is electrically and communicatively coupled to controller circuit 44 via fourth communication link 44-4. Motor 46 has a rotatable drive shaft 48. Motor 46 is directly connected to housing 43 as shown in FIG. 4.

Referring again to FIG. 1, peristaltic pump apparatus 14 further includes a first fluid reservoir 80, which has a first port 80-1; a second fluid reservoir 82, which has a second port 82-1; and a third fluid reservoir 84, which has a fifth port 84-1. After assembling the biopsy system 10, a second fluid 82-2, e.g., anesthetic, is placed inside second fluid reservoir 82. In the embodiment where there are three peristaltic pump heads, a third fluid 84-2, e.g., saline, is placed inside third fluid reservoir 84 prior to the medical procedure.

First conduit proximal end 38-2 is directly connected to first port 80-1 of first fluid reservoir 80. First conduit distal end 38-1 is directly connected to third port 30 of biopsy sampling device 12. First peristaltic pump head 50 is drivably coupled to first conduit 38. In an exemplary embodiment, the first fluid reservoir 80 is configured to receive first fluid 80-2, such as, e.g., residual saline and/or blood, from the biopsy site by pumping first fluid 80-2 present in first conduit 38 toward the proximal direction 16-2 and into first fluid reservoir 80 through first port 80-1. Pumping first conduit 38 in proximal direction 16-2 creates a vacuum in first conduit 38. Vacuum pressure may be controlled by the user of the biopsy system 10 via vacuum control button 58-1 of user interface 58.

Second conduit proximal end 40-2 is directly connected to second port 82-1 of second fluid reservoir 82. Second conduit distal end 40-1 is directly connected to fourth port 32 of biopsy sampling device 12. Second peristaltic pump head 52 is drivably coupled to second conduit 40. In an exemplary embodiment, second fluid reservoir 82 holds second fluid 82-2, e.g., anesthetic, to be pumped by second peristaltic pump head 52 in the distal direction 16-1 toward biopsy sampling device 12 upon control of the controller circuit 44 by the user interface 58 via anesthetic control button 58-2.

Third conduit proximal end 42-2 is directly connected to fifth port 84-1 of third fluid reservoir 84. Third conduit distal end 42-1 is directly connected to sixth port 34 of biopsy sampling device 12. Third peristaltic pump head 54 is drivably coupled to third conduit 42. In an exemplary embodiment, third fluid reservoir 84 holds third fluid 84-2, e.g., saline, to be pumped by third peristaltic pump head 54 toward biopsy sampling device 12 upon control of the controller circuit 44 by user interface 58 via saline control button 58-3.

In FIG. 1, first conduit 38 is shown to be arranged below first peristaltic pump head 50, second conduit 40 is show to be arranged above second peristaltic pump head 52, and third conduit 42 is shown to be arranged above third peristaltic pump head 54, so that as the rotatable drive shaft 48 is rotated in the same direction, e.g., first rotational direction 126, the following fluid paths are achieved: when first clutch assembly 60 is selected to be electromagnetically coupled to first peristaltic pump head 50, the first fluid 80-2 is pumped in a proximal direction 16-2; when second clutch assembly 62 is selected to be electromagnetically coupled to second peristaltic pump head 52, the second fluid 82-2 is pumped in the distal direction 16-1; and when third clutch assembly 64 is selected to be electromagnetically coupled to the third peristaltic pump head 54, the third fluid 84-2 is pumped in the distal direction 16-1. However, peristaltic pump apparatus 14 may have any number of peristaltic pump heads and corresponding clutch assemblies, conduits, and fluid reservoirs, and the user of the biopsy system may arrange the conduits on the peristaltic pump heads in various ways to achieve desired fluid path directions.

Turning to FIG. 4, first rotor 66 is fixedly connected to the rotatable drive shaft 48 of motor 46 via first mechanical coupler 66-1. A first stator bridge 88 connects first stator 68 to second stator 72. A stator bridge air gap 94 exists between housing 43 and first stator bridge 88. Optionally, each of the plurality of stators may be entirely independent of each other without first stator bridge 88.

Each of the plurality of stators is directly connected to housing 43 via a stator mechanical coupler 96, such as, e.g., a set screw, welding, etc. Motor 46 is directly connected to housing 43 via a motor mechanical coupler 98, such as, e.g., a set screw, welding, etc. An air gap 100 is interposed between first stator 68 and first rotor 66, so that first stator 68 remains remain stationary and first rotor 66, which is fixedly connected to the rotatable drive shaft 48, may rotate as the rotatable drive shaft 48 rotates. Air gap 100 is interposed between second stator 72 and second rotor 70, so that second stator 72 remains remain stationary and second rotor 70, which is fixedly connected to the rotatable drive shaft 48, may rotate as the rotatable drive shaft 48 rotates.

When no electrical power is delivered from electrical power source 56 to first clutch assembly 60 via first electrical connection 56-1 to first stator 68, motor 46 may receive motor instructions from controller circuit 44 via fourth communication link 44-4 to rotate rotatable drive shaft 48 and first rotor 66 will rotate about rotatable drive shaft 48 as motor 46 rotates the rotatable drive shaft 48, because first mechanical coupler 66-1 fixedly connects first rotor 66 to rotatable drive shaft 48 and because of air gap 100 between first rotor 66 and first stator 68.

First clutch assembly 60 may be selectively electromagnetically coupled to first peristaltic pump head 50 to engage first peristaltic pump head 50 in rotation about rotatable drive shaft 48. When first clutch assembly 60 is disengaged from first peristaltic pump head 50, first peristaltic pump head 50 remains stationary by way of the coupling between first peristaltic pump head 50 and rotatable drive shaft 48 even while rotatable drive shaft 48 is rotated by motor 46.

First peristaltic pump head 50 is coupled to rotatable drive shaft 48 via a first bearing assembly 101 having a first set of bearings 102 that rides inside first grooves 102-1 on the rotatable drive shaft 48. First grooves 102-1 are grooves that encircle the outer surface of rotatable drive shaft 48. First grooves 102-1 are perpendicular to the longitudinal length of rotatable drive shaft 48 as shown in FIG. 4. First set of bearings 102 may be a set of ball bearings, a set of needle bearings, or a set of bushings. First peristaltic pump head 50 has a first peristaltic pump proximal face 104 on which is a first peristaltic pump circular indention 106. First peristaltic pump circular indention 106 is circular-shaped when viewed in cross-section across the longitudinal length of rotatable drive shaft 48.

First peristaltic pump head 50 has a first spring 108 and first armature 110. First spring 108 is attached or connected inside first peristaltic pump circular indention 106. First armature 110 is made of magnetic material. First armature 110 is fixed to first spring 108. First armature 110 fits within first peristaltic pump circular indention 106 when first spring 108 is compressed. First spring 108 is compressed when first armature 110 is disengaged.

First rotor 66 has a first rotor distal face 118. First rotor 66 includes a first rotor circular indention 112 in first rotor distal face 118. First rotor circular indention 112 is circular in shape when viewed in cross-section across the longitudinal length of rotatable drive shaft 48. When first stator 68 is disengaged, an air gap 100 is interposed between first rotor circular indention 112 and first armature 110. When the processor circuit 78-1 executes first clutch assembly program instructions to energize first stator 68, first rotor 66 is magnetized and attracts first armature 110 into first rotor circular indention 112, first spring 108 extends, first armature 110 is engaged, and first armature 110 directly contacts first rotor 66. As electrical energy is supplied via first electrical connection 56-1 to first stator 68, first rotor 66 is electromagnetically coupled to first peristaltic pump head 50 and the first clutch assembly 60 is engaged. As first rotor 66 rotates with a rotation, such as, e.g., first rotational direction 126, of rotational drive shaft 48, first peristaltic pump head 50 rotates with first rotor 66 as a result of the engagement of the first clutch assembly 60 via electricity being supplied to first stator 68.

Second clutch assembly 62 and second peristaltic pump head 52 will be described. Second stator 72 is directly connected to housing 43 and is electrically connected to electrical power source 56 via second electrical connection 56-2, as shown in FIG. 2. Controller circuit 44 is electrically and communicatively coupled to the second stator 72 via second communication link 44-2, as shown in FIG. 2. Second rotor 70 is fixedly connected to the rotatable drive shaft 48 of motor 46 via a second mechanical coupler 70-1, as shown in FIG. 4.

Second peristaltic pump head 52 is coupled to rotatable drive shaft 48 via a second bearing assembly 127 having second set of bearings 128 which ride inside second grooves 128-1 on the rotatable drive shaft 48. Second grooves 128-1 are grooves that encircle the outer surface of rotatable drive shaft 48. Second grooves 128-1 are perpendicular to the longitudinal length of rotatable drive shaft 48. Second set of bearings 128 may be a set of ball bearings, a set of needle bearings, or a set of bushings.

Second peristaltic pump head 52 has a second peristaltic pump proximal face 130 on which is a second peristaltic pump circular indention 132. Second peristaltic pump circular indention 132 is circular-shaped when viewed in cross-section across the longitudinal length of rotatable drive shaft 48.

Second peristaltic pump head 52 includes a second spring 134 and a second armature 136. Second spring 134 is attached inside second peristaltic pump circular indention 132. Second armature 136 is made of magnetic material. Second armature 136 is fixed to second spring 134. Second armature 136 fits within second peristaltic pump circular indention 132 when second spring 134 is compressed. Second spring 134 is compressed when second armature 136 is disengaged.

Second rotor 70 has a second rotor distal face 138. Second rotor 70 includes a second rotor circular indention 140 in second rotor distal face 138. When second stator 72 is disengaged, an air gap 100 is interposed between second rotor circular indention 140 and second armature 136. When the processor circuit 78-1 executes second clutch assembly program instructions to energize second stator 72, second rotor 70 is magnetized to attract second armature 136 into second rotor circular indention 140, second spring 134 extends, second armature 136 is engaged, and second armature 136 directly contacts second rotor 70. As electrical energy is supplied via second electrical connection 56-2 to seconds stator 72, second rotor 70 is electromagnetically coupled to second peristaltic pump head 52 and second clutch assembly 62 is engaged. As second rotor 70 rotates with rotation, such as, e.g., first rotational direction 126 of rotational drive shaft 48, second peristaltic pump head 52 rotates with second rotor 70 as a result of the engagement of the second clutch assembly 62 via electricity being supplied to second stator 72.

Advantageously, biopsy system 10 with peristaltic pump apparatus 14 does not discharge a vacuum upon use or need repeated charging. Rather, the arrangement of first peristaltic pump head 50, first clutch assembly 60, and first conduit 38 of the peristaltic pump apparatus 14 is configured to draw a vacuum in distal sampling end 26 of cannula 20 and in sampling basket 36, and the peristaltic pump apparatus 14 is configured to deliver second fluid 82-2, such as, e.g., anesthetic, in the distal direction 16-1 toward biopsy sampling device 12 to ultimately deliver second fluid 82-2 to the target site inside the patient. The plurality of peristaltic pump heads, each of which is drivably connected to a flexible conduit, may rotate about rotatable drive shaft 48, so long as each of the plurality of peristaltic pump heads' corresponding clutch assembly is engaged. In some embodiments, a casing and clamp (not shown) couple first conduit 38 to first peristaltic pump head 50 to keep first conduit 38 from slipping off first peristaltic pump head 50.

Controller circuit 44 is configured via software and/or firmware residing in memory circuit 78-2 to execute program instructions to perform functions associated with the retrieval of biopsy tissue samples, such as that of controlling and/or monitoring one or more components of motor 46, first clutch assembly 60, second clutch assembly 62, and third clutch assembly 64. In some embodiments, controller circuit 44 is communicatively coupled to biopsy sampling device 12 and controls and monitors biopsy sampling device 12. In other embodiments, biopsy sampling device 12 has a biopsy sampling device controller circuit (not shown).

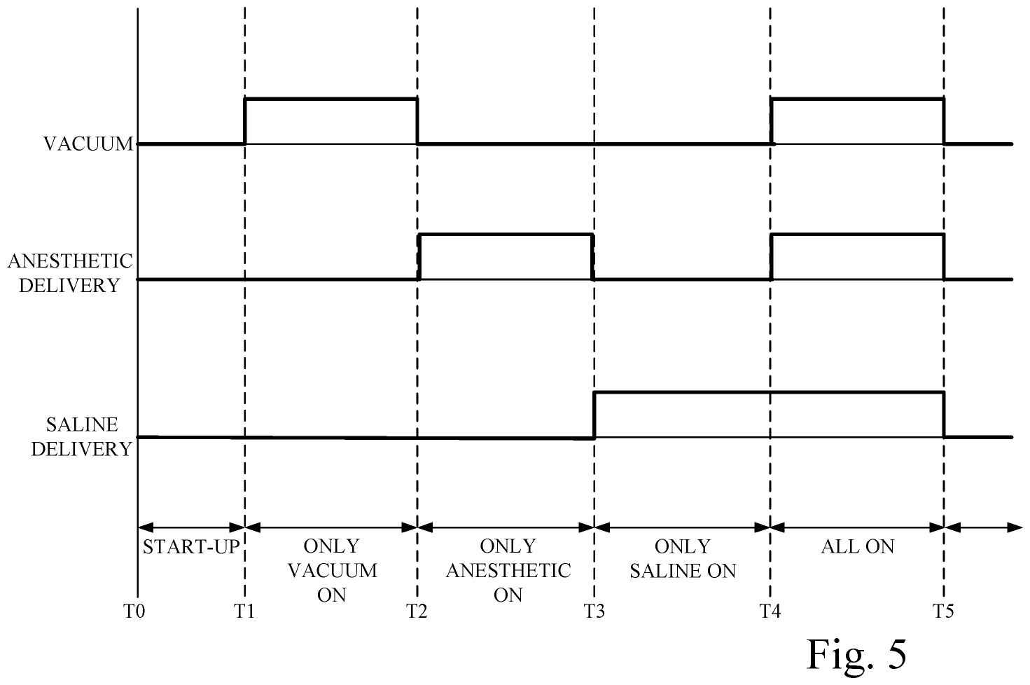

Processor circuit 78-1 is configured via software and/or firmware residing in memory circuit 78-2 to execute motor program instructions to control the rotation of rotatable drive shaft 48 of motor 46. Processor circuit 78-1 is configured via software and/or firmware residing in memory circuit 78-2 to execute first clutch assembly program instructions to selectively electromagnetically couple first rotor 66 to first peristaltic pump head 50 during a first period of engagement, e.g., from T1 to T2 in FIG. 5.

Processor circuit 78-1 is configured via software and/or firmware residing in memory circuit 78-2 to execute second clutch assembly program instructions to selectively electromagnetically couple second rotor 70 to second peristaltic pump head 52 during a second period of engagement. For example, FIG. 5 shows a second period of engagement to be between T2 and T3.

Processor circuit 78-1 is configured via software and/or firmware residing in memory circuit 78-2 to execute third clutch assembly program instructions to selectively electromagnetically couple third rotor 74 to third peristaltic pump head 54 during a third period of engagement. For example, FIG. 4 shows a third period of engagement to be between T3 and T5.

Applicant's use of the ordinal numbers “first”, “second” and “third” to describe the various periods of engagement are not to indicate order or sequence of a method, but instead are used as nomenclature only. The order of application of the various periods of engagement is entirely at the discretion of the user of the peristaltic pump apparatus. Furthermore, the term “first period of engagement” refers to any period of time in which the processor circuit 78-1 executes first clutch assembly instructions to select the first clutch assembly 60 to be engaged so that the first rotor 66 is electromagnetically coupled to the first peristaltic pump head 50. The term “second period of engagement” refers to any period of time in which the processor circuit 78-1 executes second clutch assembly instructions to select the second clutch assembly 62 to be engaged so that the second rotor 70 is electromagnetically coupled to the second peristaltic pump head 52. The term “third period of engagement” refers to any period of time in which the processor circuit 78-1 executes third clutch assembly instructions to select the third clutch assembly 64 to be engaged so that the third rotor 74 is electromagnetically coupled to the third peristaltic pump head 54.

Controller circuit 44 is configured via software and/or firmware residing in memory circuit 78-2 to execute program instructions to Pulse Width Modulate (PWM) the electrical control signals to each clutch assembly, more specifically, each stator. Controller circuit 44 is configured via the processor circuit to execute the first clutch assembly program instructions to deliver a first electrical control signal, which may be pulse width modulated, to the first clutch assembly 60 and, in particular, to the first stator 68, to control a first speed of rotation of the first peristaltic pump head 50. Controller circuit 44 is configured via the processor circuit 78-1 to execute the second clutch assembly program instructions to deliver a second electrical control signal, which may be pulse width modulated, to the second clutch assembly 62 and, in particular, to the second stator 72, to control a second speed of rotation of the second peristaltic pump head 52. Controller circuit 44 is configured via the processor circuit 78-1 to execute the third clutch assembly program instructions to deliver a third electrical control signal, which may be pulse width modulated, to the third clutch assembly 64 and, in particular, to the third stator 76, to control a third speed of rotation of the third peristaltic pump head 54. The first speed of rotation is correlated with a first fluid flow rate of the first clutch assembly 60. The first fluid flow rate may be measured as vacuum pressure. The second speed of rotation is correlated with a second fluid flow rate of the second clutch assembly 62. The third speed of rotation is correlated with a third fluid flow rate of the third clutch assembly 64. The controller circuit 44 is configured to independently control the first clutch assembly 60, the second clutch assembly 62, and the third clutch assembly 64. Through Pulse Width Modulation, controller circuit 44 is able to independently control the individual speed of rotation of each peristaltic pump head, e.g., first peristaltic pump head 50. Pulse Width Modulation effectively engages and disengages the first clutch assembly 60 to control the first fluid flow rate, which may be measured as vacuum pressure, independent of the second fluid flow rate of the second clutch assembly 62 and the third fluid flow rate of the third clutch assembly 64.

Controller circuit 44 is configured to apply Pulse Width Modulation to the first period of engagement to control the vacuum pressure. If a maximum flow rate is chosen for the first peristaltic pump head 50, controller circuit 44 will send the first electrical control signal over first communication link 44-1 to not apply Pulse Width Modulation to the electricity supplied over first electrical connection 56-1 to the first clutch assembly 60, more specifically, to first stator 68, and a 100% duty cycle will be supplied via first electrical connection 56-1 to first stator 68.

Furthermore, controller circuit 44 is configured to control the frequency of electrical pulse in the electrical control signals being sent to each of first stator 68, second stator 72, and third stator 76.

The user of the biopsy system 10 may manually control the vacuum pressure in first conduit 38 via vacuum control button 58-1 and first communication link 44-1 to control the rate of proximal return of first fluid 80-2 to first fluid reservoir 80. As the user sets the rate of proximal return of first fluid 80-2, controller circuit 44 translates that information into a PWM duty cycle and a frequency to control the first clutch assembly 60.

The user of biopsy system 10 may manually control the anesthetic flow rate of second fluid 82-2 in second conduit 40 via anesthetic control button 58-2 and second communication link 44-2 to second stator 72. As the user sets the second fluid delivery rate of second fluid 82-2, controller circuit 44 translates that information into a PWM duty cycle and a frequency to control the second clutch assembly 62.

The user of the biopsy system 10 may manually control the saline flow rate of third fluid 84-2 in third conduit 42 via saline control button 58-3 and third communication link 44-3 to third stator 76. As the user sets the rate of proximal return of third fluid 84-2, controller circuit 44 translates that information into a PWM duty cycle and a frequency to control the third clutch assembly 64.

FIG. 5 is a line graph depicting the delivery of various fluids, more specifically, a vacuum, anesthetic, and saline, by an exemplary embodiment of the peristaltic pump apparatus 14 over time. According to the exemplary embodiment of FIG. 5, the vacuum is delivered in the distal direction 16-1 by the first peristaltic pump head 50, which has the effect of delivering the first fluid 80-2, e.g., residual saline, in the proximal direction 16-2 during a first period of engagement. The first period of engagement is shown in FIG. 5 to be from T1 to T2, which is labeled “ONLY VACUUM ON”, and repeated from T4 to T5, which is labeled “ALL ON”.

Furthermore, FIG. 5 graphically shows delivery of second fluid 82-2, e.g., anesthetic, by the second peristaltic pump head 52 during a second period of engagement. The second period of engagement is shown in FIG. 5 to be from T2 to T3, which is labeled “ONLY ANESTHETIC ON”, and from T4 to T5, which is labeled “ALL ON”.

Moreover, FIG. 5 shows delivery of the third fluid 84-2, e.g., saline, by the third peristaltic pump head 54 during a third period of engagement. The third period of engagement is shown to be from T3 to T5. A portion of the third period of engagement is shown from T3 to T4 and is labeled “ONLY SALINE ON” and a second portion of the third period of engagement is shown from T4 to T5 as “ALL ON”.

The first period of engagement, second period of engagement, and third period of engagement are repeatable at regular or irregular intervals. FIG. 5 demonstrates the first period of engagement repeats from T4 to T5 and the second period of engagement repeats from T4 to T5.

In an exemplary embodiment, the first period of engagement may be longer than the second period of engagement. For example, FIG. 5 shows the third period of engagement is longer than either the first period of engagement or the second period of engagement. In an exemplary embodiment, first period of engagement and second period of engagement may have identical durations, but the first period of engagement and the second period of engagement do not start on the same start time, as shown in FIG. 5.

In an exemplary embodiment, the first period of engagement and the second period of engagement are not identical and do not overlap. For instance, FIG. 5 shows that the vacuum is delivered at T1 and turned off at T2 while the anesthetic delivery is turned on at T2, so that the first period of engagement does not overlap the second period of engagement between T1 and T3.

In an exemplary embodiment, the first period of engagement and the second period of engagement are not identical, but overlap. For example, in FIG. 5 the third period of engagement, shown from T3 to T5, has a longer duration than the first period of engagement, repeated from T4 to T5, and the second period of engagement, repeated from T4 to T5, but the first period of engagement, the second period of engagement, and the third period of engagement overlap from T4 to T5.

The first period of engagement begins upon a first start time, e.g., T1, and the second period of engagement begins upon a second start time, e.g., T2. In some embodiments, the first start time and the second start time are identical. For example, in FIG. 5, the first period of engagement is repeated with a first start time at T4 and the second period of engagement is repeated with a second start time at T4.

In an exemplary embodiment, a first start time and a second start time differ, and the first period of engagement and the second period of engagement overlap. For example, FIG. 5 shows the third period of engagement begins at T3, differing from T4, which is the second start time of the repeated second period of engagement, and from T4 to T5 the third period of engagement and the second period of engagement overlap.

Furthermore, in the exemplary embodiment shown in FIG. 5, first start time T1 and second start time T2 differ, and first period of engagement, shown from T1 to T2, and second period of engagement, shown from T2 to T3, do not coincide or overlap.

The controller circuit 44 is configured to engage each of the first stator 68, the second stator 72, and the third stator 76 in any order and entirely selectively and independently. For example, an operator of the biopsy system 10 may choose to engage the third clutch assembly 64 to generate the third period of engagement, e.g., saline delivery to the biopsy site, before engaging the first clutch assembly 60 to produce the first period of engagement, e.g., vacuum pressure at the biopsy site. For another example, an operator of the biopsy system 10 may choose to engage the second clutch assembly 62 to deliver the second fluid 82-2, e.g., anesthetic, to the biopsy site and to keep the third clutch assembly 64 not engaged so as to prevent dilution of the second fluid 82-2 at the biopsy site.

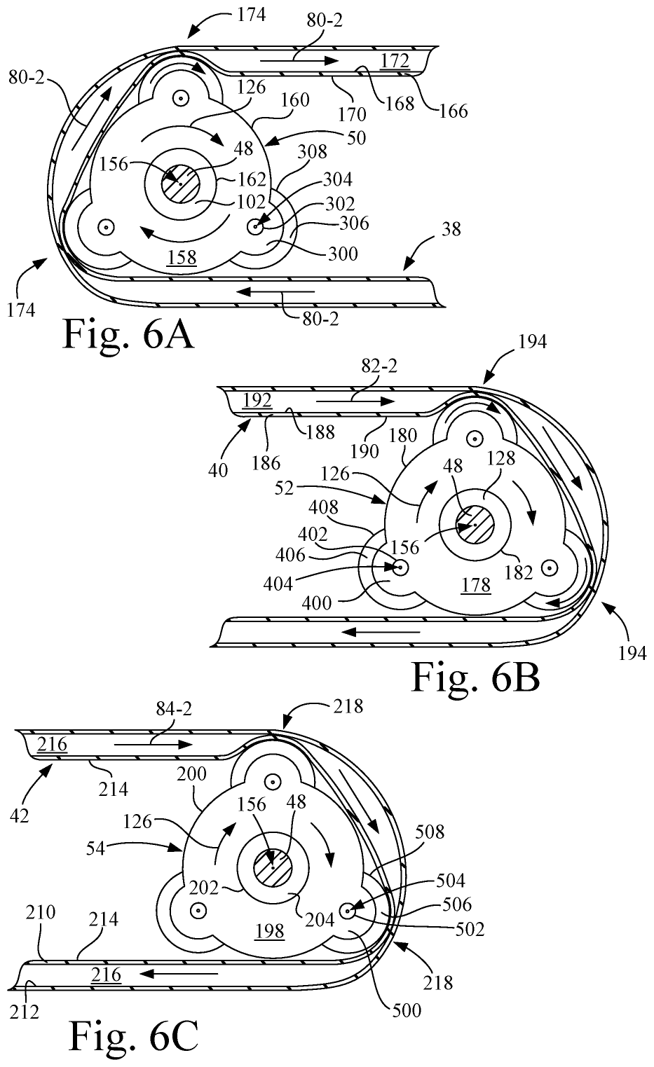

FIG. 6A shows a cross-section taken along line 6A in FIG. 3 of first peristaltic pump head 50. First peristaltic pump head 50 has a first body 158, a first outermost perimeter 160 on the outside of first body 158, and a first aperture 162 that is centrally located inside the first body 158. First peristaltic pump head 50 is generally cylindric in form. First peristaltic pump head 50 is configured to rotate about a central axis 156 upon electromagnetic coupling of first rotor 66 to first peristaltic pump head 50. First aperture 162 is a cylindrically-shaped void that is centrally positioned on central axis 156. First set of bearings 102 fit inside first aperture 162 and contact the outer surface of rotatable drive shaft 48. As rotatable drive shaft 48 rotates, first set of bearings 102 rotate within first aperture 162 while first peristaltic pump head 50 remains stationary.

FIG. 6A shows that first conduit 38 has a first conduit side wall 166. First conduit side wall 166 has a first conduit interior surface 168 and a first conduit exterior surface 170. First conduit interior surface 168 is configured to define a first conduit lumen 172 therein.

First peristaltic pump head 50 includes at least three first projections 300 that are angularly spaced around first outermost perimeter 160 of first body 158. Centered in each of the at least three first projections 300 is a first projection aperture 302. Inside each first projection aperture 302 is a first axle 304 that runs parallel to rotatable drive shaft 48. A first idle roller 306 is rotatably mounted on each first axle 304 inside each of the at least three first projections 300. FIG. 6A shows three first idle rollers 306 connected to the first peristaltic pump head 50 and the three first idle rollers 306 are angularly spaced around first outermost perimeter 160 of first body 158.

At least two of the at least three first idle rollers 306 make contact with first conduit exterior surface 170 at any given point of time. As first peristaltic pump head 50 rotates with the rotatable drive shaft 48 when the first clutch assembly 60 is engaged, at least two of the at least three first idle rollers 306 are directly contacting first conduit 38, as shown in FIG. 6A. Pressure created by a first contact point 174 between first idle roller 306 and first conduit 38 causes first conduit interior surface 168 to contact the opposite side of the first conduit lumen 172, which is also first conduit interior surface 168. Contact between first idle roller 306 and first conduit 38 causes first conduit lumen 172 to collapse and effectively create a valve, albeit momentarily, as first peristaltic pump head 50 rotates with rotatable drive shaft 48 when first clutch assembly 60 is engaged during the first period of engagement, e.g., from T1 to T2 in FIG. 5. Moreover, the addition of the at least three first idle rollers 306 to the first outermost perimeter 160 configures first peristaltic pump head 50 to pump first fluid 80-2 inside first conduit lumen 172 and maintains first conduit 38 in a stationary position.

Advantageously, in some embodiments, each first idle roller 306 is configured with a first idle roller surface 308 for receiving first conduit 38. First idle roller surface 308 is configured with a surface depression or groove to receive first conduit 38 to keep first conduit 38 from slipping off of first idle roller 306 as first peristaltic pump head 50 and first idle roller 306 rotates during engagement of first clutch assembly 60. The electrical power source 56 is not configured to supply electricity directly to first idle roller 306.

FIG. 6B shows a cross-section taken along line 6B in FIG. 3 of an embodiment of second peristaltic pump head 52. Second peristaltic pump head 52 has a second body 178, a second outermost perimeter 180, and a second aperture 182 that is centrally located inside the second body 178. Second peristaltic pump head 52 is generally cylindric in form. Second peristaltic pump head 52 is configured to rotate about central axis 156 in a first rotational direction 126 upon the electromagnetic coupling of the second rotor 70 to the second peristaltic pump head 52. Second aperture 182 is a cylindrically-shaped void that is centrally positioned on second body 178.

Second peristaltic pump head 52 is coupled to rotatable drive shaft 48 via second set of bearings 128. Second set of bearings 128 may be a second set of ball bearings, a second set of needle bearings, or a second set of bushings, each of which is known to a person of ordinary skill in the art. Second set of bearings 128 fit inside second aperture 182 and are configured to move within second grooves 128-1 that surround the outer surface of rotatable drive shaft 48. Thus, as rotatable drive shaft 48 rotates, second set of bearings 128 rotate within second aperture 182 while second peristaltic pump head 52 remains stationary. FIG. 6B shows three second idle rollers 406 connected to the second peristaltic pump head 52 and the three second idle rollers 406 are angularly spaced around second outermost perimeter 180 of second body 178.

FIG. 6B shows second conduit 40 has a second conduit side wall 186. Second conduit side wall 186 has a second conduit interior surface 188 and a second conduit exterior surface 190. Second conduit interior surface 188 is configured to define a second conduit lumen 192 therein.

Second peristaltic pump head 52 includes at least three second projections 400 that are angularly spaced around second body 178. Centered in each of the at least three second projections 400 is a second projection aperture 402. Inside each second projection aperture 402 is a second axle 404 that runs parallel to rotatable drive shaft 48. A second idle roller 406 is rotatably mounted on each second axle 404 inside each of the at least three second projections 400.

At least two of the at least three second idle rollers 406 make contact with second conduit exterior surface 190 at any given point of time. As second peristaltic pump head 52 rotates with the rotatable drive shaft 48 during engagement of second clutch assembly 62, at least two of the at least three second idle rollers 406 are directly contacting second conduit 40. Pressure created by the contact at second contact point 194 between any of the at least three second idle rollers 406 and second conduit 40 causes second conduit interior surface 188 to contact the opposite side of the second conduit lumen 192, which is also second conduit interior surface 188. The contact between second idle roller 406 and second conduit 40 causes second conduit lumen 192 to collapse and effectively create a valve, albeit momentarily, as second peristaltic pump head 52 rotates with rotatable drive shaft 48 about the axis of rotatable drive shaft 48 when second clutch assembly 62 is engaged during second period of engagement, e.g. from T2 to T3 in FIG. 5. Moreover, the addition of the at least three second idle rollers 406 to the second outermost perimeter 180 configures second peristaltic pump head 52 to pump second fluid 82-2 inside second conduit lumen 192 and maintains second conduit 40 in a stationary position.

Advantageously, in some embodiments, each second idle roller 406 is configured with a second idle roller surface 408 for receiving second conduit 40. The second idle roller surface 408 is configured with a surface depression or groove to receive second conduit 40 to keep second conduit 40 from slipping off of second idle roller 406 as the second peristaltic pump head 52 and second idle roller 406 rotates during engagement of second clutch assembly 62. No electrical power is supplied directly to second idle roller 406.

FIG. 6C shows a cross-section taken along line 6C in FIG. 3 of third peristaltic pump head 54. Third peristaltic pump head 54 has a third body 198, a third outermost perimeter 200 on the outside of third body 198, and a third aperture 202 that is centrally located inside the third body 198. Third peristaltic pump head 54 is generally cylindric in form. Third peristaltic pump head 54 is configured to rotate about central axis 156 upon electromagnetic coupling of third rotor 74 to third peristaltic pump head 54. Third aperture 202 is a cylindrically-shaped void that is centrally positioned on central axis 156. Third peristaltic pump head 54 is coupled to rotatable drive shaft 48 via a third set of bearings 204. Third set of bearings 204 may be a third set of ball bearings, a third set of needle bearings, or a third set of bushings, each of which is known to a person of ordinary skill in the art. Third set of bearings 204 fit inside third aperture 202 and are configured to move within third grooves 206 (not shown) that surround the outer surface of rotatable drive shaft 48. Thus, as rotatable drive shaft 48 rotates, third set of bearings 204 rotate within third aperture 202 while third peristaltic pump head 54 remains stationary.

FIG. 6C shows that third conduit 42 has a third conduit side wall 210 that has a third conduit interior surface 212 and a third conduit exterior surface 214. Third conduit interior surface 212 is configured to define a third conduit lumen 216 therein.

Third peristaltic pump head 54 includes at least three third projections 500 that are angularly spaced around the third body 198. Centered in each of the at least three third projections 500 is a third projection aperture 502. Inside each third projection aperture 502 is a third axle 504 that runs parallel to rotatable drive shaft 48. A third idle roller 506 is rotatably mounted on each third axle 504 inside each of the at least three third projections 500. FIG. 6C shows three third idle rollers 506 connected to the third peristaltic pump head 54 and the three third idle rollers 506 are angularly spaced around third outermost perimeter 200 of third body 198.

At least two of the at least three third idle rollers 506 make contact with third conduit exterior surface 214 at any given point of time. As third peristaltic pump head 54 rotates with the rotatable drive shaft 48, at least two of the at least three third idle rollers 506 are directly contacting third conduit 42. Pressure created by a third contact point 218 between third idle roller 506 and third conduit 42 causes third conduit interior surface 212 to contact the opposite side of the third conduit lumen 216, which is also third conduit interior surface 212. Contact between third idle roller 506 and third conduit 42 causes third conduit lumen 216 to collapse and effectively create a valve, albeit momentarily, as third peristaltic pump head 54 rotates with rotatable drive shaft 48 when third clutch assembly 64 is engaged during third period of engagement, e.g. from T3 to T5 in FIG. 5.

Advantageously, in some embodiments, each third idle roller 506 is configured with a third idle roller surface 508 for receiving third conduit 42. Third idle roller surface 508 is configured with a surface depression or groove to receive third conduit 42 to keep third conduit 42 from slipping off of third idle roller 506 as third peristaltic pump head 54 and third idle roller 506 rotates during engagement of third clutch assembly 64. No electrical power is supplied directly to third idle roller 506.

First peristaltic pump head 50, first conduit 38, and first fluid reservoir 80 are arranged to prevent a positive pressure fluid flow in distal direction 16-1 toward the biopsy sampling device 12. First peristaltic pump head 50, first conduit 38, and first fluid reservoir 80 are arranged so that as controller circuit 44 has first rotor 66 engage with first peristaltic pump head 50 and controller circuit 44 has the motor 46 rotate rotatable drive shaft 48 a vacuum is created in first conduit 38 that flows in the proximal direction 16-2 toward the first fluid reservoir 80. Given that first conduit distal end 38-1 is directly connected to third port 30 of biopsy sampling device 12 and third port 30 is proximal sampling basket 36 of biopsy sampling device 12, the vacuum created by the arrangement of first peristaltic pump head 50, first conduit 38, and first fluid reservoir 80 extends to sampling basket 36 and distal sampling end 26 of biopsy sampling device 12. Any sampled tissue that is received in the distal sampling end 26 of biopsy sampling device 12 is suctioned into the sampling basket 36. First fluid reservoir 80 collects any fluids removed from the biopsy target site that filter through the sampling basket 36. In an exemplary embodiment, first fluid reservoir 80 is disposable.

Peristaltic pump apparatus 14 includes a vacuum in first conduit 38 and second fluid 82-2 in second conduit 40. First peristaltic pump head 50 is configured to drive first fluid 80-2 in first conduit 38 in the proximal direction 16-2 toward first fluid reservoir 80 during the first period of engagement, e.g. from T1 to T2 in FIG. 5. Moreover, there are no valves on first conduit 38 (other than first contact point 174). At the outset, first peristaltic pump head 50 is configured to begin pumping air through first conduit 38, which is a condition known as a dry start. Second peristaltic pump head 52 is configured to drive second fluid 82-2 in second conduit 40 in the distal direction 16-1 toward fourth port 32 during the second period of engagement, e.g. from T2 to T3 in FIG. 5.

The peristaltic pump apparatus 14 is configured so that the delivery of first fluid 80-2 in the proximal direction 16-2, the delivery of second fluid 82-2 in the distal direction 16-1, and the delivery of third fluid 84-2 in the distal direction 16-1 are independently controlled by way of the controller circuit 44 having separate and individual communication links 44-1, 44-2, and 44-3, respectively, to each of first clutch assembly 60, second clutch assembly 62, and third clutch assembly 64.

Furthermore, the user of the exemplary peristaltic pump apparatus 14 may direct controller circuit 44 to deliver only anesthetic to biopsy sampling device 12 and, thus, to the sampling site by activating the controller circuit 44 to electromagnetically couple the second rotor 70 to the second peristaltic pump head 52, and the user may choose to uncouple other rotors from their respective peristaltic pump heads. The user may also decide to deliver only third fluid 84-2 to biopsy sampling device 12 by selecting (or pressing) saline control button 58-3 (whether physical buttons or a touchscreen icon) on the user interface 58 to direct the controller circuit 44 to electromagnetically couple the third rotor 74 to the third peristaltic pump head 54 and to uncouple other rotors from their respective peristaltic pump heads. The user may choose to provide a vacuum via first conduit 38 to sampling basket 36 and to deliver second fluid 82-2 via second conduit 40 to biopsy sampling device 12 by directing the controller circuit 44 to electromagnetically couple the first rotor 66 to the first peristaltic pump head 50, electromagnetically couple the second rotor 70 to the second peristaltic pump head 52, and to uncouple the third rotor 74 from the third peristaltic pump head 54. Furthermore, the peristaltic pump apparatus 14 is configured such that a user may choose to direct the controller circuit 44 to deliver electrical control signals to each of the plurality of clutch assemblies to electromagnetically couple each of the plurality of clutch assemblies to each respective corresponding peristaltic pump head to cause all three functions of the exemplary embodiment of the peristaltic pump apparatus 14 to be performed simultaneously.

An advantage of using peristaltic pump apparatus 14 over a traditional vacuum pump is that the rotation, such as, e.g., first rotational direction 126, of the rotatable drive shaft 48 may be increased or decreased by the user of the biopsy system 10 to vary the amount of suction present at the biopsy sampling device 12. Some tissues do not require a strong vacuum. In an exemplary embodiment, after all of the conduits are assembled in the peristaltic pump apparatus 14 and connected to the appropriate ports, the user may operate peristaltic pump apparatus 14 to create a vacuum in the first conduit 38, which will in turn create a vacuum in distal sampling end 26 to expose sampling notch 28. When a sample is ready to be taken, the user will select the proper button on user interface 58 on a console or a handheld device to cause motor 46 to rotate rotatable drive shaft 48. Upon start up, neither first clutch assembly 60 nor second clutch assembly 62 are engaged, meaning first rotor 66 is not electromagnetically coupled to first peristaltic pump head 50 and second rotor 70 is not electromagnetically coupled to second peristaltic pump head 52. However, motor 46 will continuously be rotating rotatable drive shaft 48 upon start up at T0, shown in FIG. 5.

The number of revolutions per minute (RPM) may be set on a default setting or increased or decreased by the user's control of the controller circuit 44 via user interface 58. The faster the rotatable drive shaft 48 rotates, the stronger the suction of the tissue sample through the cannula 20. After the tissue sample is cut, the tissue sample is transported in the proximal direction 16-2 to sampling basket 36. Cannula 20 and sampling basket 36 are removable and disposable from the rest of biopsy sampling device 12 to prevent cross-contamination between samples taken at different tissue sites or from other patients. The tissue sample is removed from the sampling basket 36 for further analysis.

Furthermore, the exemplary biopsy system 10 also reduces auditory loudness, complexity, and procedural time. The exemplary biopsy system 10 is less complex, because there is no need for valve assemblies to charge and discharge the system vacuum.

As used herein the terms “substantially”, “generally”, “slightly”, and other words of degree are relative modifiers intended to indicate permissible variation from the characteristic so modified. Such terms are not intended to be limited to the absolute value of the characteristic which it modifies, but rather possessing more of the physical or functional characteristic than the opposite, and approaching or approximating such a physical or functional characteristic.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A peristaltic pump apparatus for use with a medical device, comprising:
a housing;
a motor having a rotatable drive shaft, the motor directly connected to the housing;
a first peristaltic pump head coupled to the rotatable drive shaft;
a second peristaltic pump head coupled to the rotatable drive shaft;
a first clutch assembly having a first rotor fixedly connected to the rotatable drive shaft and a first stator directly connected to the housing, the first clutch assembly configured to selectively electromagnetically couple the first rotor to the first peristaltic pump head; and
a second clutch assembly having a second rotor fixedly connected to the rotatable drive shaft and a second stator directly connected to the housing, the second clutch assembly configured to selectively electromagnetically couple the second rotor to the second peristaltic pump head.

2. The peristaltic pump apparatus of claim 1, comprising a controller circuit electrically coupled to each of the motor, the first clutch assembly, and the second clutch assembly, the controller circuit configured to selectively supply electrical control signals to each of the motor, the first clutch assembly, and the second clutch assembly.

3. The peristaltic pump apparatus of claim 2, wherein the controller circuit has a processor circuit and a memory circuit, the processor circuit is configured to execute motor program instructions to control the rotation of the rotatable drive shaft of the motor, the processor circuit is configured to execute first clutch assembly program instructions to selectively electromagnetically couple the first rotor to the first peristaltic pump head during a first period of engagement, and the processor circuit is configured to execute second clutch assembly program instructions to selectively electromagnetically couple the second rotor to the second peristaltic pump head during a second period of engagement.

4. The peristaltic pump apparatus of claim 3, wherein the first period of engagement and the second period of engagement are not identical.

5. The peristaltic pump apparatus of claim 3, wherein the first period of engagement and the second period of engagement are repeatable at regular intervals.

6. The peristaltic pump apparatus of claim 3, wherein the first period of engagement begins upon a first start time and the second period of engagement begins upon a second start time, the first start time differs from the second start time.

7. The peristaltic pump apparatus of claim 6, wherein the first start time and the second start time are not identical, the first period of engagement and the second period of engagement do not overlap.

8. The peristaltic pump apparatus of claim 6, wherein the first start time and the second start time are not identical, the first period of engagement and the second period of engagement overlap.

9. The peristaltic pump apparatus of claim 1, wherein the first peristaltic pump head is coupled to the rotatable drive shaft via a first set of bearings, the second peristaltic pump head is coupled to the rotatable drive shaft via a second set of bearings.

10. The peristaltic pump apparatus of claim 1, wherein the first rotor is fixedly connected to the rotatable drive shaft via a first mechanical coupler, the second rotor is fixedly connected to the rotatable drive shaft via a second mechanical coupler.

11. The peristaltic pump apparatus of claim 1, comprising:
the first peristaltic head having a first outermost perimeter;
the second peristaltic head having a second outermost perimeter;
at least three first idle rollers connected to the first peristaltic pump head, the at least three first idle rollers are angularly spaced around the first outermost perimeter; and
at least three second idle rollers connected to the second peristaltic pump head, the at least three second idle rollers are angularly spaced around the second outermost perimeter.

12. The peristaltic pump apparatus of claim 3, wherein the controller circuit is configured to generate a first speed of rotation of the first peristaltic pump head via a first electrical control signal, the controller circuit is configured to generate a second speed of rotation of the second peristaltic pump head via a second electrical control signal, the first speed of rotation is correlated with a first fluid flow rate of the first clutch assembly, the second speed of rotation is correlated with a second fluid flow rate of the second clutch assembly, the controller circuit is configured to independently control the first clutch assembly and the second clutch assembly.

13. A biopsy system comprising:
a biopsy sampling device; and
a peristaltic pump apparatus coupled in fluid communication with the biopsy sampling device, the peristaltic pump apparatus comprising:
a housing;
a controller circuit;
a motor having a rotatable drive shaft, the motor electrically coupled to the controller circuit, the motor directly connected to the housing;
a first peristaltic pump head coupled to the rotatable drive shaft;
a second peristaltic pump head coupled to the rotatable drive shaft;
a first clutch assembly having a first rotor fixedly connected to the rotatable drive shaft and a first stator directly connected to the housing, the first clutch assembly electrically coupled to the controller circuit, the first clutch assembly configured to selectively electromagnetically couple the first rotor to the first peristaltic pump head; and
a second clutch assembly having a second rotor fixedly connected to the rotatable drive shaft and a second stator directly connected to the housing, the second clutch assembly electrically coupled to the controller circuit, the second clutch assembly configured to selectively electromagnetically couple the second rotor to the second peristaltic pump head,
wherein the controller circuit is configured to selectively supply electrical control signals to each of the motor, the first clutch assembly, and the second clutch assembly.

14. The biopsy system of claim 13, wherein the controller circuit has a processor circuit and a memory circuit, the processor circuit is configured to execute motor program instructions to control the rotation of the rotatable drive shaft of the motor, the processor circuit is configured to execute first clutch assembly program instructions to selectively electromagnetically couple the first rotor to the first peristaltic pump head during a first period of engagement, and the processor circuit is configured to execute second clutch assembly program instructions to selectively electromagnetically couple the second rotor to the second peristaltic pump head during a second period of engagement.

15. The biopsy system of claim 14, the peristaltic pump apparatus comprising:
a first fluid reservoir having a first port;
a second fluid reservoir having a second port;
a first conduit having a first conduit distal end and a first conduit proximal end, the first conduit proximal end directly connected to the first port; and
a second conduit having a second conduit distal end and a second conduit proximal end, the second conduit proximal end directly connected to the second port,
wherein the first peristaltic pump head, the first conduit, and the first fluid reservoir are arranged to prevent a positive pressure fluid flow in a distal direction.

16. The biopsy system of claim 15, comprising:
the biopsy sampling device having a cannula, a third port, a fourth port, and a sampling basket, the cannula having a distal sampling end, the sampling basket disposed between the distal sampling end of the cannula and the third port, the sampling basket is distal of the third port, the fourth port disposed between the distal sampling end of the cannula and the sampling basket, the fourth port is distal of the third port; and
the peristaltic pump apparatus comprising:
a first fluid in the first conduit;
a second fluid in the second conduit;
the first peristaltic pump head is drivably coupled to the first conduit;
the second peristaltic pump head is drivably coupled to the second conduit;
the first conduit distal end is directly connected to the third port; and
the second conduit distal end is directly connected to the fourth port,
wherein the first peristaltic pump head is configured to drive the first fluid in the first conduit in a proximal direction toward the first fluid reservoir during the first period of engagement, the second peristaltic pump head is configured to drive the second fluid in the second conduit in the distal direction toward the fourth port during the second period of engagement.

17. The biopsy system of claim 15, wherein there are no valves on the first conduit, the first peristaltic pump assembly is configured to begin pumping the first conduit with a dry start.

18. The biopsy system of claim 15, the peristaltic pump apparatus comprising:
the first peristaltic head having a first outermost perimeter;
the second peristaltic head having a second outermost perimeter;
at least three first idle rollers connected to the first peristaltic pump head, the at least three first idle rollers are angularly spaced around the first outermost perimeter; and
at least three second idle rollers connected to the second peristaltic pump head, the at least three second idle rollers are angularly spaced around the second outermost perimeter,
wherein at least two of the at least three first idle rollers on the first peristaltic pump head are in direct contact with the first conduit and at least two of the at least three second idle rollers on the second peristaltic pump head are in direct contact with the second conduit.

19. A biopsy system comprising:
a biopsy sampling device having a cannula, a third port, a fourth port, and a sampling basket, the cannula having a distal sampling end, the sampling basket disposed between the distal sampling end of the cannula and the third port, the sampling basket is distal of the third port, the fourth port disposed between the distal sampling end of the cannula and the sampling basket, the fourth port is distal of the third port; and
a peristaltic pump apparatus coupled in fluid communication with the biopsy sampling device, the peristaltic pump apparatus comprising:
a housing;
a controller circuit;
a motor having a rotatable drive shaft, the motor electrically coupled to the controller circuit, the motor directly connected to the housing;
a first fluid reservoir having a first port;
a second fluid reservoir having a second port;
a first conduit having a first conduit distal end and a first conduit proximal end, the first conduit proximal end directly connected to the first port, the first conduit distal end directly connected to the third port;
a second conduit having a second conduit distal end and a second conduit proximal end, the second conduit proximal end directly connected to the second port, the second conduit distal end directly connected to the fourth port;
a first peristaltic pump head coupled to the rotatable drive shaft, the first peristaltic head having a first outermost perimeter;
a second peristaltic pump head coupled to the rotatable drive shaft, the second peristaltic pump head having a second outermost perimeter;
at least three first idle rollers connected to the first peristaltic pump head, the at least three first idle rollers are angularly spaced around the first outermost perimeter;
at least three second idle rollers connected to the second peristaltic pump head, the at least three second idle rollers are angularly spaced around the second outermost perimeter;
a first clutch assembly having a first rotor fixedly connected to the rotatable drive shaft and a first stator directly connected to the housing, the first clutch assembly electrically coupled to the controller circuit, the first clutch assembly configured to selectively electromagnetically couple the first rotor to the first peristaltic pump head; and
a second clutch assembly having a second rotor fixedly connected to the rotatable drive shaft and a second stator directly connected to the housing, the second clutch assembly electrically coupled to the controller circuit, the second clutch assembly configured to selectively electromagnetically couple the second rotor to the second peristaltic pump head;
wherein
the controller circuit has a processor circuit and a memory circuit, the processor circuit is configured to execute motor program instructions to control the rotation of the rotatable drive shaft of the motor, the processor circuit is configured to execute first clutch assembly program instructions to selectively electromagnetically couple the first rotor to the first peristaltic pump head during a first period of engagement, and the processor circuit is configured to execute second clutch assembly program instructions to selectively electromagnetically couple the second rotor to the second peristaltic pump head during a second period of engagement,
at least two of the at least three first idle rollers contact the first conduit during the first period of engagement,
at least two of the at least three second idle rollers contact the second conduit during the second period of engagement, and
the first peristaltic pump head, the first conduit, and the first fluid reservoir are arranged to prevent a positive pressure fluid flow in a distal direction and to create a vacuum that flows in a proximal direction toward the first fluid reservoir.

20. The biopsy system of claim 19, wherein the peristaltic pump apparatus comprises a first fluid in the first conduit and a second fluid in the second conduit, wherein the first peristaltic pump head is configured to drive the first fluid in the first conduit in the proximal direction toward the first fluid reservoir during the first period of engagement, the second peristaltic pump head is configured to drive the second fluid in the second conduit in the distal direction toward the fourth port during the second period of engagement.
